(19) United States
(12) Patent Application Publication
(10) Pub. No.: US 2025/0188021 A1
(43) Pub. Date: Jun. 12, 2025

(54) PREPARATION METHOD FOR ALICYCLIC CARBAMATE AND USE THEREOF

(71) Applicant: Institute of Process Engineering, Chinese Academy of Sciences, Beijing (CN)

(72) Inventors: Liguo WANG, Beijing (CN); Shuang XU, Beijing (CN); Huiquan LI, Beijing (CN); Peng HE, Beijing (CN); Yan CAO, Beijing (CN); Zheng ZHENG, Beijing (CN); Jiaqiang CHEN, Beijing (CN)

(73) Assignee: Institute of Process Engineering, Chinese Academy of Sciences, Beijing (CN)

(21) Appl. No.: 18/844,333

(22) PCT Filed: Jul. 26, 2022
(86) PCT No.: PCT/CN2022/107861

§ 371 Date: Sep. 5, 2024

(30) Foreign Application Priority Data
Mar. 7, 2022 (CN) 202210216513.0

(51) Int. Cl. C07C 269/06; B01J 21/04; B01J 21/08; B01J 23/46; B01J 23/63; B01J 23/89; B01J 27/24; C08G 71/04

(52) U.S. Cl. CPC C07C 269/06; B01J 21/04; B01J 21/08; B01J 23/462; B01J 23/63; B01J 23/892; B01J 27/24; C08G 71/04

(57) ABSTRACT

Disclosed herein are a preparation method for an alicyclic carbamate and the use thereof. The preparation method comprises the following steps: mixing a carbamate containing an aromatic ring, a catalyst and a solvent, and then introducing hydrogen for reaction to obtain the alicyclic carbamate, wherein the catalyst comprises a carrier and an active component, which is loaded on the carrier, the active component comprising any one or a combination of at least two of Pt, Rh, Ru, Ir or Pd. The preparation method for the alicyclic carbamate provided in the present application has mild reaction conditions, easy operation, low potential safety hazard and a high yield of the alicyclic carbamate, is suitable for multiple reactors, is easy for large-scale continuous production, and has good industrial application prospects.

### TECHNICAL FIELD

Embodiments of the present application relate to the field of chemical industry, and especially, a preparation method for an alicyclic carbamate and use thereof, such as a high-yield preparation method for an alicyclic carbamate and use thereof.

### BACKGROUND

The production technologies of polyurethane have become increasingly fully developed, the most mature of which is the isocyanate route. Polyurethane materials prepared from aliphatic and alicyclic diisocyanate (short as ADI) have excellent mechanical properties, outstanding chemical stability and excellent weather resistance, and are widely used in fields such as high-grade coatings, high-grade synthetic leather, elastomers, adhesives, and rocket propellants. Compared with aromatic isocyanates such as MDI and TDI, most species of ADI series have better performance and lower toxicity.

Alicyclic diisocyanate belong to ADI, and its synthesis methods are mainly divided into the phosgene method and non-phosgene method. The phosgene method is commonly used in the industry, but the phosgene used in this method for preparing isocyanates is highly toxic, and a large amount of hydrogen chloride will be generated during the reaction, which may cause equipment corrosion and environmental pollution, and thus this method is highly dangerous and difficult to operate. From the perspective of green and environmental protection, it is necessary to develop non-phosgene technology. The non-phosgene method is mainly the carbamate splitting method. Carbamate is the key precursor for isocyanate synthesis by pyrolysis. Additionally, polyurethane can be directly synthesized by transesterification of carbamate and polyol, which is a non-isocyanate route to synthesize polyurethane.

In the current technical routes, for both phosgene method and non-phosgene method, it is necessary to hydrogenate the benzene ring of aromatic ring-containing diamine to obtain alicyclic diamine first, and then the non-phosgene or phosgene method is used to synthesize ADI.

In order to inhibit the side reaction of deamination (or de-methylamine) of aromatic ring-containing amine during the benzene-ring hydrogenation, it is often needed to modify the catalyst by alkalization, or add promoters such as alkali metal salts and nitrite, or add ammonia and organic amines, so as inhibit the side reaction of deamination (or de-methylamine). However, the loss of alkali increases the regeneration frequency of the catalyst and increases the cost; the introduction of a large amount of ammonia in industrial devices will lead to equipment corrosion and bring safety risks; the recycling cost of organic amine solvent is high; continuously adding new promoters during the use of the catalyst will lead to constant accumulation residual of alkali metal and impair the catalyst performance.

CN110105223A discloses that m-xylylenediamine is mixed with the solvent and cocatalyst and prepared into a mixed solution; 1,3-cyclohexanebis (methylamine) is continuously prepared by a fixed bed reactor; however, the cocatalyst contains nitrate or nitrite.

U.S. Pat. No. 3,697,449A1 discloses that 1-35 wt % alkali metal of alkoxide or an aqueous solution of hydroxide is used to modify the supported ruthenium catalyst, and then hydrogenation reduction reaction of diaminodiphenylmethane is carried out.

CN111804324A discloses that amino lithium is used to modify the metal-supported catalyst, which is used to catalyze the hydrogenation of diaminodiphenylmethane, avoiding secondary amine by-products and the increase of PACM-OH. However, lithium is easy to be lost during the use process, which increases the cost of product after-treatment and catalyst regeneration.

In view of the adverse effects of alkali modification or alkali metal salt, nitrite, ammonia or organic amine in the process of synthesizing alicyclic amine by direct benzene-ring hydrogenation of aromatic ring-containing amines, as well as the environmental and safety problems of synthesizing isocyanate and polyurethane by the phosgene method, it is of great significance to develop a green and safe synthetic route of alicyclic carbamate.

### SUMMARY

The following is a summary of the subject matter described in detail herein. This summary is not intended to limit the protection scope of the claims.

An embodiment of the present application provides a preparation method for an alicyclic carbamate and use thereof, and especially a high-yield preparation method for an alicyclic carbamate and use thereof. The preparation method for an alicyclic carbamate provided by the present application has the advantages of mild reaction conditions, simple operation, low potential safety risk, high yield of the alicyclic carbamate, suitability for various reactors, easiness for large-scale continuous production, and good industrial application prospect.

In one aspect, an embodiment of the present application provides a preparation method for an alicyclic carbamate, which comprises the following steps: mixing an aromatic ring-containing carbamate, a catalyst and a solvent, and then performing a reaction with hydrogen introduced to obtain the alicyclic carbamate.

The catalyst comprises a carrier and an active component loaded on the carrier.

The active component comprises any one or a combination of at least two of Pt, Rh, Ru, Ir or Pd, such as a combination of Pt and Rh, a combination of Rh and Ru, or a combination of Ir and Pd, but the active component is not limited to the above-mentioned combinations, and other unlisted combinations within the combination range are also applicable.

The preparation method avoids the problems of by-products from deamination condensation occurring in direct hydrogenation of aromatic ring-containing amines, and catalyst deactivation caused by tar, and catalyst component loss. The product alicyclic carbamate is an important intermediate for the synthesis of ADI by non-phosgene method, and provides a low-toxic, safe and green production process for the synthesis of ADI. At the same time, the yield of the product can be effectively improved by selecting particular catalysts.

Preferably, the active component is any one of a combination of Ru and Pd, a combination of Rh and Ru, a combination of Ru and Pt, a combination of Ru and Ir, a combination of Rh and Pt, a combination of Ir and Pt, a combination of Pd and Pt or a combination of Ir and Pd, preferably a combination of Ru and Pd.

The above specific active component further improves the yield of the product.

Preferably, the carrier comprises any one or a combination of at least two of SiO₂, Al₂O₃, ZrO₂, TiO₂, MgO, kaolin, bentonite, montmorillonite, ZSM-5, X-zeolite, Y-zeolite, B-zeolite, mordenite, spinel, magnesium-aluminum hydrotalcite, activated carbon, graphene, carbon nanotubes, g-C₃N₄ (graphitic carbon nitride), h-BN (hexagonal boron nitride) or a nitrogen-doped carbon composite, such as a combination of SiO₂ and Al₂O₃, a combination of X-zeolite and Y-zeolite or a combination of activated carbon and graphene, but the carrier is not limited to the above-mentioned combinations, and other unlisted combinations within the combination range are also applicable.

Preferably, the catalyst further comprises an auxiliary agent loaded on the carrier, and the auxiliary agent comprises an elemental metal and/or a metal oxide.

Preferably, the elemental metal comprises any one or a combination of at least two of Ni, Fe, Co, La or Ce, such as a combination of Ni and Fe, a combination of Co and La, or a combination of La and Ce, but the elemental metal is not limited to the above-mentioned combinations, and other unlisted combinations within the combination range are also applicable.

Preferably, the auxiliary agent is any one or a combination of at least two of Ni, Fe, Co, La₂O₃, CeO₂, NiO, Ni₂O₃, FeO, Fe₂O₃, Fe₃O₄, CoO, Co₂O₃ or Co₃O₄, preferably a combination of Ni and Co.

The selection of the specific auxiliary agent can further improve the reaction efficiency and improve the yield of product.

Preferably, a mass of the active component is 0.1-10% of the total mass of the catalyst.

Preferably, a mass of the auxiliary agent is 0-10% of the total mass of the catalyst, and 0 means that the catalyst does not contain the auxiliary agent.

Preferably, a mass ratio of the aromatic ring-containing carbamate to the catalyst is (30-100):0.1.

The mass of the active component may be 0.1%, 0.2%, 0.3%, 0.5%, 1%, 2%, 3%, 4%, 5%, 6%, 7%, 8%, 9% or 10% of the total mass of the catalyst, the mass of the auxiliary agent may be 0.1%, 0.2%, 0.3%, 0.5%, 1%, 2%, 3%, 4%, 5%, 6%, 7%, 8%, 9% or 10% of the total mass of the catalyst, and the mass ratio of the aromatic ring-containing carbamate to the catalyst may be 30:0.1, 40:0.1, 50:0.1, 60:0.1, 70:0.1, 80:0.1, 90:0.1 or 100:0.1; however, the features are not limited to the listed values, and other unlisted values within the above numerical ranges are also applicable.

Preferably, the solvent comprises any one or a combination of at least two of methanol, water, ethanol, n-propanol, isopropanol, n-butanol, 2-butanol, tetrahydrofuran, dimethylformamide, dimethyl sulfoxide, 2-methyltetrahydrofuran, 1,4-dioxane, ethyl acetate, dimethyl carbonate, diethyl carbonate, ethyl methyl carbonate, methylcyclohexane or cyclohexane, such as a combination of methanol and water, a combination of ethanol and n-propanol, or a combination of dimethyl carbonate and diethyl carbonate, but the solvent is not limited to the above-mentioned combinations, and other unlisted combinations within the combination range are also applicable. The solvent is preferably any one of a combination of methanol and water, a combination of methanol and ethanol, a combination of ethanol and water, a combination of tetrahydrofuran and water, a combination of tetrahydrofuran and ethanol, and a combination of tetrahydrofuran and methanol, and further preferably a combination of ethanol and water.

The specific solvent can effectively improve the reaction efficiency, and at the same time, the yield of product can be further improved by using the preferred solvent combination.

Preferably, the aromatic ring-containing carbamate has a general formula as follows:

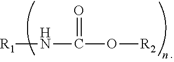

In the general formula, R₁ is selected from a substituted or unsubstituted C6-C30 aromatic ring-containing hydrocarbyl group, and a substitute or unsubstituted aryl group; substituted substituents are selected from any one of a nitro group, a hydroxyl group, an alkylmercapto group, an arylmercapto group, a sulfonyl group, a carbonyl group, a halogen atom, a cyano group, an amino group, a carboxyl group, an ester group, an alkoxy group or an aryloxy group, and the aryl group and aromatic ring are independently selected from a benzene ring, biphenyl, naphthalene or diphenylmethane;
n is an integer selected from 1 to 5, such as 1, 2, 3, 4 or 5; and
R₂ is selected from a linear or branched C1-C8 saturated hydrocarbyl group or a C5-C10 saturated cyclic hydrocarbyl group.

The above C6-C30 respectively indicate that the structure contains six carbon atoms, seven carbon atoms, eight carbon atoms, nine carbon atoms . . . , and so on, which is not listed exhaustedly here, and the similar interpretation is suitable for the C1-C8 and C5-C10.

Preferably, R₂ is selected from any one of a methyl group, an ethyl group, a n-propyl group, an isopropyl group, a n-butyl group, an isobutyl group, a sec-butyl group, a pentyl group, a hexyl group, a heptyl group, an octyl group, an isooctyl group, a 2-ethylhexyl group, a cyclopentyl group or a cyclohexyl group.

It should be noted that the aromatic ring-containing hydrocarbyl group contains at least one benzene ring, and the aromatic hydrocarbyl group can be bonded with an aliphatic hydrocarbyl group. In this case, the amino group in the aromatic ring-containing carbamate can be directly bonded to the aromatic hydrocarbon, or it can be bonded to the aliphatic hydrocarbyl group on the aromatic hydrocarbon, both of which are feasible.

The aromatic ring-containing carbamate may have the structure, for example, as shown in Formula I to Formula IX (not limited to the examples):

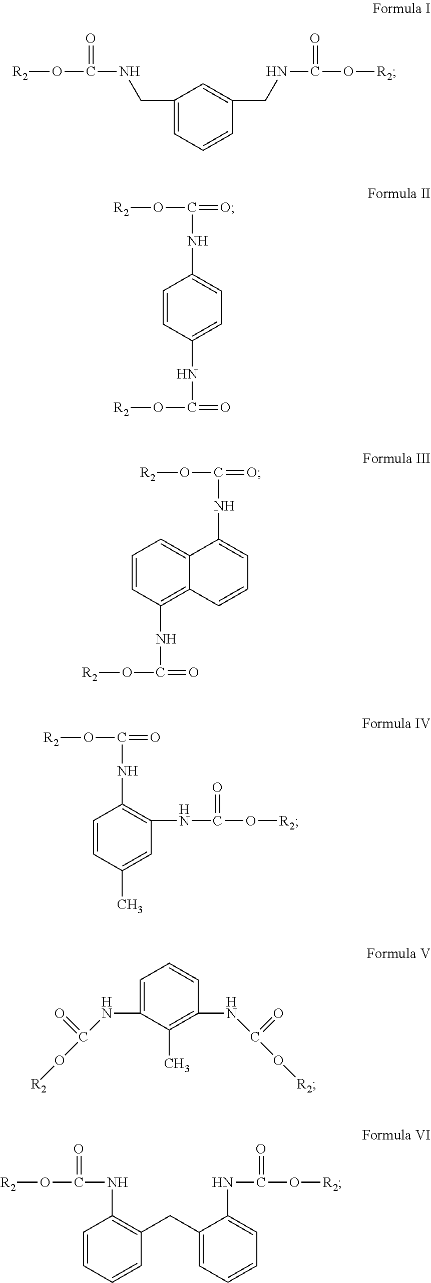

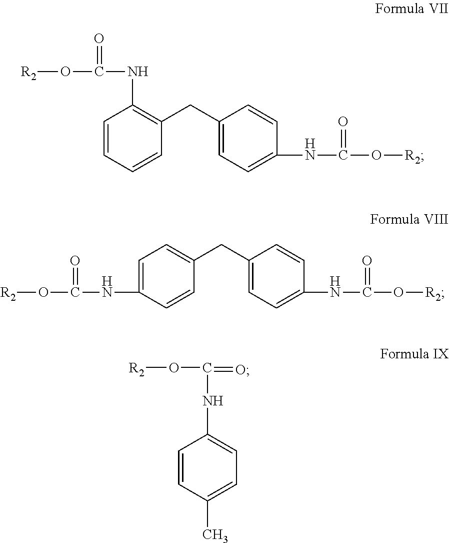

but the aromatic ring-containing carbamate is not limited to the compounds listed above, wherein R₂ has the same definition as described above.

Preferably, a mass fraction of the aromatic ring-containing carbamate is 0.5-30% in the solvent, such as 0.5%, 1%, 3%, 6%, 9%, 12%, 15%, 18%, 21%, 24%, 27% or 30%, but the mass fraction is not limited to the listed values, and other unlisted values within the above numerical range are also applicable.

Preferably, the reaction is performed in a reactor, and the reactor is any one of a fixed bed, a fluidized bed or a tank reactor.

Preferably, the reactor is a fixed bed or a fluidized bed, the reaction is performed at a temperature of 0-180° C., the reaction is performed at a pressure of 0.1-10 MPa, a molar ratio of the hydrogen to the aromatic ring-containing carbamate is (20-300):1, and a liquid hourly space velocity of the aromatic ring-containing carbamate is 0.1-10 h⁻¹.

The temperature of the reaction may be 0° C., 20° C., 40° C., 60° C., 80° C., 100° C., 120° C., 140° C., 160° C. or 180° C., the pressure of the reaction may be 0.1 MPa, 0.2 MPa, 0.3 MPa, 0.5 MPa, 1 MPa, 2 MPa, 3 MPa, 4 MPa, 5 MPa, 6 MPa, 7 MPa, 8 MPa, 9 MPa or 10 MPa, the molar ratio of the hydrogen to the aromatic ring-containing carbamate may be 20:1, 40:1, 60:1, 80:1, 100:1, 120:1, 140:1, 160:1, 180:1, 200:1, 220:1, 240:1, 260:1, 280:1, or 300:1, and the liquid hourly space velocity of the aromatic ring-containing carbamate may be 0.1 h⁻¹, 0.2 h⁻¹, 0.3 h⁻¹, 0.5 h⁻¹, 1 h⁻¹, 2 h⁻¹, 3 h⁻¹, 4 h⁻¹, 5 h⁻¹, 6 h⁻¹, 7 h⁻¹, 8 h⁻¹, 9 h⁻¹ or 10 h⁻¹; however, the features are not limited to the listed values, and other unlisted values within the above numerical ranges are also applicable.

Preferably, the reactor is a tank reactor, the reaction is performed at a temperature of 0-180° C., and the reaction is performed at a pressure of 0.1-10 MPa.

The temperature of the reaction may be 0° C., 20° C., 40° C., 60° C., 80° C., 100° C., 120° C., 140° C., 160° C. or 180° C., and the pressure of the reaction may be 0.1 MPa, 0.2 MPa, 0.3 MPa, 0.5 MPa, 1 MPa, 2 MPa, 3 MPa, 4 MPa, 5 MPa, 6 MPa, 7 MPa, 8 MPa, 9 MPa or 10 MPa; however, the features are not limited to the listed values, and other unlisted values within the above numerical ranges are also applicable.

In another aspect, the present application provides use of the preparation method for an alicyclic carbamate as described above in the preparation of polyurethane.

Compared with the related art, the present application has the following beneficial effects:
an embodiment of the present application provides a preparation method for an alicyclic carbamate; the preparation method avoids the problems of by-products from deamination condensation occurring in direct hydrogenation of aromatic ring-containing amines, and catalyst deactivation caused by tar, and catalyst component loss; the product alicyclic carbamate is an important intermediate for the synthesis of ADI by non-phosgene method, and provides a low-toxic, safe, and green production process for the synthesis of ADI; at the same time, the yield of product can be effectively improved by selecting particular catalysts, and the yield of product can be further improved by selecting particular auxiliary agents and solvents.

Other aspects can be understood after reading and appreciating the detailed description.

### DETAILED DESCRIPTION

In order to further illustrate the technical solutions adopted in the present application and their effects, the technical scheme of the present application will be further explained in terms of the preferred embodiments of the present application, but the present application is not limited to the scope of the embodiments.

### Example 1

This example provides a preparation method for an alicyclic carbamate. The method specifically comprises the following steps:

2 g of 0.5% Pd-0.5% Ru-0.1% Co-0.1% Ni/Al₂O₃ was weighed out and added into a fixed bed reactor of 600 mm length and 10 mm inner diameter; a reaction solvent was ethanol and water (with a volume ratio of 1:1), a mass fraction of diethyl phenylene-1,4-dicarbamate (as shown in Formula II) was 5% in the solvent, a reaction temperature was 30° C., a reaction pressure was 2.0 MPa, a molar ratio of H₂:diethyl phenylenedicarbamate was 100:1, and a liquid hourly space velocity of diethyl phenylenedicarbamate was 0.2 h⁻¹; after the reaction was stable, sample analysis showed that a conversion rate of diethyl phenylenedicarbamate was 99.5% and a yield of diethyl cyclohexane-1,4-dicarbamate was 99.3%.

### Example 2

This example provides a preparation method for an alicyclic carbamate. The method specifically comprises the following steps:

2 g of 3% Ru/SiO₂ was weighed out and added into a fixed bed reactor of 600 mm length and 10 mm inner diameter; a reaction solvent was ethanol, a mass fraction of dimethyl (phenylene-1,3-dimethylene)dicarbamate (as shown in Formula I) was 20% in the solvent, a reaction temperature was 130° C., a reaction pressure was 2.0 MPa, a molar ratio of H₂:diethyl phenylenedicarbamate was 100:1, and a liquid hourly space velocity of diethyl phenylenedicarbamate was 0.5 h⁻¹; after the reaction was stable, sample analysis showed that a conversion rate of diethyl (1,3-phenylenedimethylene)dicarbamate was 99.6% and a yield of dimethyl (cyclohexane-1,3-diylbis(methylene))dicarbamate was 99.4%.

### Example 3

This example provides a preparation method for an alicyclic carbamate. The method specifically comprises the following steps:

2 g of 1% Ru/g-C₃N₄ was weighed out and added into a fixed bed reactor of 600 mm length and 10 mm inner diameter; a reaction solvent was tetrahydrofuran, a mass fraction of diethyl 1,4-phenylenedicarbamate (as shown in Formula II) was 10% in the solvent, a reaction temperature was 160° C., a reaction pressure was 5.0 MPa, a molar ratio of H₂:diethyl phenylenedicarbamate was 200:1, and a liquid hourly space velocity of diethyl phenylenedicarbamate was 2.0 h⁻¹; after the reaction was stable, sample analysis showed that a conversion rate of diethyl phenylenedicarbamate was 99.3% and a yield of diethyl cyclohexane-1,4-dicarbamate was 99.1%.

### Example 4

This example provides a preparation method for an alicyclic carbamate. The method specifically comprises the following steps:

0.6 g of 1% Ru/g-C₃N₄, 12 g of methyl 4-methylphenylcarbamate (as shown in Formula IX), and 228 g of solvent methanol were weighed out and fed into a 500 mL stainless steel autoclave; after replace the air in the autoclave by nitrogen, hydrogen was introduced, stirring was started, a reaction temperature was controlled at 60° C., a reaction pressure was 3.0 MPa, and the reaction was performed for 1 h and then stopped; sample analysis showed that a conversion rate of methyl 4-methylphenylcarbamate was 99.6% and a yield of methyl 4-methylcyclohexyldicarbamate was 99.3%.

### Example 5

This example provides a preparation method for an alicyclic carbamate, the steps of which are the same as those of Example 1 except that the catalyst was replaced with 0.5% Pd-0.5% Ru/Al₂O₃ by an equal amount.

Finally, a conversion rate of diethyl phenylenedicarbamate was 92.5% and a yield of diethyl cyclohexane-1,4-dicarbamate was 90.3%.

### Example 6

This example provides a preparation method for an alicyclic carbamate, the steps of which are the same as those of Example 1 except that the catalyst was replaced with 0.5% Pd-0.5% Pt-0.1% Co-0.1% Ni/Al₂O₃ by an equal amount.

Finally, a conversion rate of diethyl phenylenedicarbamate was 93.4% and a yield of diethyl cyclohexane-1,4-dicarbamate was 90.5%.

### Example 7

This example provides a preparation method for an alicyclic carbamate, the steps of which are the same as those of Example 1 except that the catalyst was replaced with 0.5% Rh-0.5% Ru-0.1% Co-0.1% Ni/Al₂O₃ by an equal amount.

Finally, a conversion rate of diethyl phenylenedicarbamate was 91.8% and a yield of diethyl cyclohexane-1,4-dicarbamate was 90.3%.

### Example 8

This example provides a preparation method for an alicyclic carbamate, the steps of which are the same as those of Example 1 except that the catalyst was replaced with 0.5% Pd-0.5% Ru-0.1% Co-0.1% Fe/Al₂O₃ by an equal amount.

Finally, a conversion rate of diethyl phenylenedicarbamate was 94.5% and a yield of diethyl cyclohexane-1,4-dicarbamate was 93.6%.

### Example 9

This example provides a preparation method for an alicyclic carbamate, the steps of which are the same as those of Example 1 except that the catalyst was replaced with 0.5% Pd-0.5% Ru-0.2% CeO₂/Al₂O₃ by an equal amount.

Finally, a conversion rate of diethyl phenylenedicarbamate was 95.5% and a yield of diethyl cyclohexane-1,4-dicarbamate was 94.8%.

### Example 10

This example provides a preparation method for an alicyclic carbamate, the steps of which are the same as those of Example 1 except that the solvent was replaced with methanol by an equal amount.

Finally, a conversion rate of diethyl phenylenedicarbamate was 88.9% and a yield of diethyl cyclohexane-1,4-dicarbamate was 88.3%.

### Example 11

This example provides a preparation method for an alicyclic carbamate, the steps of which are the same as those of Example 1 except that the solvent was replaced with a mixed solvent of cyclohexane and methanol (with a volume ratio of 1:1) by an equal amount.

Finally, a conversion rate of diethyl phenylenedicarbamate was 93.5% and a yield of diethyl cyclohexane-1,4-dicarbamate was 92.5%.

The above results show that the preparation method for an alicyclic carbamate provided by the present application is adaptable to various reactors, with high reaction conversion rate and high yield; Comparing Examples 1, 5-11, it can be found that the conversion rate and yield of the reaction of the present application are further improved by using the combination of specific active components, auxiliary agents and solvents.

The applicant declared that the present application illustrates the preparation method for an alicyclic carbamate and the use thereof of the present application through the above-mentioned examples, but the present application is not limited to the above-mentioned examples, that is, the present application does not necessarily rely on the above-mentioned examples for implementation. It should be clear to those skilled in the field that any improvements to the present application, equivalent replacement of raw materials, addition of auxiliary components, and selection of specific methods of the present application shall all fall within the protection scope and disclosure scope of the present application.

The preferred embodiments of the present application have been described in detail above, but the present application is not limited to the specific details in the above-mentioned embodiments. Under the technical concepts of the present application, the technical scheme of the present application may have lots of simple variations, and these simple variations shall all be covered by the protection scope of the present application.

In addition, it should be noted that the specific technical features described in the above-mentioned embodiments can be combined in any suitable way as long as there is no contradiction. In order to avoid unnecessary repetition, the possible combination methods will not be explained in the present application.

What is claimed is:

1. A preparation method for an alicyclic carbamate, comprising the following steps: mixing an aromatic ring-containing carbamate, a catalyst and a solvent, and then performing a reaction with hydrogen introduced to obtain the alicyclic carbamate;
the catalyst comprises a carrier and an active component loaded on the carrier; the the active component comprises any one or a combination of at least two of Pt, Rh, Ru, Ir or Pd.

2. The preparation method for an alicyclic carbamate according to claim 1, wherein the active component is any one of a combination of Ru and Pd, a combination of Rh and Ru, a combination of Ru and Pt, a combination of Ru and Ir, a combination of Rh and Pt, a combination of Ir and Pt, a combination of Pd and Pt or a combination of Ir and Pd, preferably a combination of Ru and Pd.

3. The preparation method for an alicyclic carbamate according to claim 1, wherein the carrier comprises any one or a combination of at least two of SiO₂, Al₂O₃, ZrO₂, TiO₂, MgO, kaolin, bentonite, montmorillonite, ZSM-5, X-zeolite, Y-zeolite, B-zeolite, mordenite, spinel, magnesium-aluminum hydrotalcite, activated carbon, graphene, carbon nanotubes, g-C₃N₄, h-BN or a nitrogen-doped carbon composite.

4. The preparation method for an alicyclic carbamate according to claim 1, wherein the catalyst further comprises an auxiliary agent loaded on the carrier, and the auxiliary agent comprises an elemental metal and/or a metal oxide.

5. The preparation method for an alicyclic carbamate according to claim 4, wherein the elemental metal comprises any one or a combination of at least two of Ni, Fe, Co, La or Ce;
preferably, the auxiliary agent is any one or a combination of at least two of Ni, Fe, Co, La₂O₃, CeO₂, NiO, Ni₂O₃, FeO, Fe₂O₃, Fe₃O₄, CoO, Co₂O₃ or Co₃O₄, preferably a combination of Ni and Co.

6. The preparation method for an alicyclic carbamate according to claim 1, wherein a mass of the active component is 0.1-10% of the total mass of the catalyst;
preferably, a mass of the auxiliary agent is 0-10% of the total mass of the catalyst, and 0 means that the catalyst does not contain the auxiliary agent;
preferably, a mass ratio of the aromatic ring-containing carbamate to the catalyst is (30-100):0.1.

7. The preparation method for an alicyclic carbamate according to claim 1, wherein the solvent comprises any one or a combination of at least two of methanol, water, ethanol, n-propanol, isopropanol, n-butanol, 2-butanol, tetrahydrofuran, dimethylformamide, dimethyl sulfoxide, 2-methyltetrahydrofuran, 1,4-dioxane, ethyl acetate, dimethyl carbonate, diethyl carbonate, ethyl methyl carbonate, methylcyclohexane or cyclohexane, and preferably a combination of methanol and water, a combination of methanol and ethanol, a combination of ethanol and water, a combination of tetrahydrofuran and water, a combination of tetrahydrofuran and ethanol, and a combination of tetrahydrofuran and methanol, and further preferably a combination of ethanol and water.

8. The preparation method for an alicyclic carbamate according to claim 1, wherein the aromatic ring-containing carbamate has a general formula as follows: 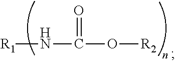
wherein R₁ is selected from a substituted or unsubstituted C6-C30 aromatic ring-containing hydrocarbyl group, and a substitute or unsubstituted aryl group; substituted substituents are selected from any one of a nitro group, a hydroxyl group, an alkylmercapto group, an arylmercapto group, a sulfonyl group, a carbonyl group, a halogen atom, a cyano group, an amino group, a carboxyl group, an ester group, an alkoxy group or an aryloxy group, and the aryl group and aromatic ring are independently selected from a benzene ring, biphenyl, naphthalene or diphenylmethane;
n is an integer selected from 1 to 5; and
R₂ is selected from a linear or branched C1-C8 saturated hydrocarbyl group or a C5-C10 saturated cyclic hydrocarbyl group.

9. The preparation method for an alicyclic carbamate according to claim 8, wherein R₂ is selected from any one of a methyl group, an ethyl group, a n-propyl group, an isopropyl group, a n-butyl group, an isobutyl group, a sec-butyl group, a pentyl group, a hexyl group, a heptyl group, an octyl group, an isooctyl group, a 2-ethylhexyl group, a cyclopentyl group or a cyclohexyl group.

10. The preparation method for an alicyclic carbamate according to claim 1, wherein a mass fraction of the aromatic ring-containing carbamate is 0.5-30% in the solvent.

11. The preparation method for an alicyclic carbamate according to claim 1, wherein the reaction is performed in a reactor, and the reactor is any one of a fixed bed, a fluidized bed or a tank reactor.

12. The preparation method for an alicyclic carbamate according to claim 11, wherein the reactor is a fixed bed or a fluidized bed, the reaction is performed at a temperature of 0-180° C., the reaction is performed at a pressure of 0.1-10 MPa, a molar ratio of the hydrogen to the aromatic ring-containing carbamate is (20-300):1, and a liquid hourly space velocity of the aromatic ring-containing carbamate is 0.1-10 h⁻¹.

13. The preparation method for an alicyclic carbamate according to claim 11, wherein the reactor is a tank reactor, the reaction is performed at a temperature of 0-180° C., and the reaction is performed at a pressure of 0.1-10 MPa.

14. Use of the preparation method for an alicyclic carbamate according to claim 1 in the preparation of polyurethane.
